(19) United States
(12) Patent Application Publication
(10) Pub. No.: US 2023/0065004 A1
(43) Pub. Date: Mar. 2, 2023

(54) METHOD FOR IMPROVING COLOUR RETENTION OF DYED KERATINOUS MATERIAL, ESPECIALLY HUMAN HAIR

(71) Applicant: Henkel AG & Co. KGaA, Duesseldorf (DE)

(72) Inventors: Constanze KRUCK, Grevenbroich (DE); Sandra HILBIG, Bochum (DE); Melanie MOCH, Dormagen (DE); Susanne DICKHOF, Viersen (DE); Daniela KESSLER-BECKER, Leverkusen (DE)

(73) Assignee: Henkel AG & Co. KGaA, Duesseldorf (DE)

(21) Appl. No.: 17/780,372

(22) PCT Filed: Sep. 14, 2020
(86) PCT No.: PCT/EP2020/075594

§ 371 Date: May 26, 2022

(30) Foreign Application Priority Data
Nov. 26, 2019 (DE) 10 2019 218 228.3

(51) Int. Cl. A61K 8/898; A61Q 5/00; A61K 8/19; A45D 19/00

(52) U.S. Cl. CPC A61K 8/898; A61Q 5/004; A61K 8/19; A45D 19/0066; A61K 2800/4324; A45D 2200/25

(57) ABSTRACT

An objective of the present disclosure is a process for improving the color retention on keratinous material which has been colored by application of at least one pigment, wherein an aftertreatment agent is applied to the colored keratinous material and, if necessary, is rinsed off again after a reaction time, wherein the aftertreatment agent is
(N-1) Water comprises,
(N-2) has a pH value of 7.0 to 12.5, and
(N-3) comprises at least one salt of a divalent cation.

### CROSS-REFERENCE TO RELATED APPLICATION

This application is a U.S. National-Stage entry under 35 U.S.C. § 371 based on International Application No. PCT/EP2020/075594, filed Sep. 14, 2020, which was published under PCT Article 21(2) and which claims priority to German Application No. 102019218228.3, filed Nov. 26, 2019, which are all hereby incorporated in their entirety by reference.

### TECHNICAL FIELD

The objective of the present application is a process for improving the color retention of keratinous material previously colored by application of at least one pigment, wherein an aftertreatment agent is applied to the colored keratinous material and, if necessary, rinsed off again after a reaction time. Here, the aftertreatment agent is wherein it comprises water, has a pH of about 7.0 to about 12.5 and comprises at least one salt of a divalent cation

A second subject of this application is a process for dyeing and after treating keratinous fibers, in particular human hair, in which first a dyeing agent comprising at least one amino silicone and at least one pigment is applied, and then the after treating agent described above is applied.

A third subject of the present application is a multi-component packaging unit (kit-of-parts), which comprises the previously described colorant and the after-treatment agent in separately packaged containers.

### BACKGROUND

Changing the shape and color of keratinous material, especially human hair, is a key area of modern cosmetics. To change the hair color, the expert knows various coloring systems depending on the coloring requirements. Oxidation dyes are usually used for permanent, intensive dyeings with good fastness properties and good grey coverage. Such colorants contain oxidation dye precursors, so-called developer components and coupler components, which, under the influence of oxidizing agents such as hydrogen peroxide, form the actual dyes among themselves. Oxidation dyes are exemplified by very long-lasting dyeing results.

When direct dyes are used, ready-made dyes diffuse from the colorant into the hair fiber. Compared to oxidative hair dyeing, the dyeings obtained with direct dyes have a shorter shelf life and quicker wash ability. Dyes with direct dyes usually remain on the hair for a period of between 5 and 20 washes.

The use of color pigments is known for short-term color changes on the hair and/or skin. Pigments or color pigments are understood to be insoluble, color-imparting substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

If the user wants particularly long-lasting dyeings, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair. A continuing challenge is therefore the search for alternative, high-performance dyeing processes. One viable alternative coloring system that has recently come increasingly into focus is based on the use of colored pigments.

Coloring with pigments offers several significant advantages. Since the pigments only attach themselves to the keratin materials, especially the hair fibers, from the outside, the damage associated with the dyeing process is particularly low. Furthermore, colorations that are no longer desired can be removed quickly and easily without leaving any residue, thus offering the user the possibility of returning to his original hair color immediately and without significant effort. Especially for those consumers who do not want to recolor their hair regularly, this coloring process is therefore particularly attractive.

In recent work, the problem of low durability of this staining system has been addressed. In this context, it was found that the wash fastness of the color results obtained with pigments could be improved by combining the pigments with certain amino-functionalized silicone polymers. Despite the possibilities thus found to improve wash fastness or color retention, there is still a need for optimization in this context. For this reason, possibilities are still being sought to further improve a pigment-based dyeing system in terms of its wash fastness or color retention.

### BRIEF SUMMARY

This disclosure provides a process for improving the color retention on keratinous material which has been colored by the application of at least one pigment, wherein an after treatment agent is applied to the colored keratinous material and optionally rinsed off after a reaction time, wherein the after treatment agent comprises
(N-1) Water,
(N-2) has a pH value of about 7.0 to about 12.5, and
(N-3) further comprises at least one salt of a divalent cation.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has been the object of the present disclosure to provide a staining system having fastness properties comparable to oxidative staining, if possible. Wash fastness properties should be outstanding, but the use of oxidation dye precursors normally used for this purpose should be avoided. A technology was sought that would make it possible to fix the colorant compounds (especially pigments) known from the prior art to the hair in an extremely durable manner. When the agents are used in a dyeing process, intensive dyeing results with good fastness properties and good color retention should be obtained.

Surprisingly, it has now been found that keratinic materials previously dyed by the application of amino silicones and pigments have particularly good wash fastness or color retention when an alkaline, water-comprising aftertreatment agent comprising at least one salt of a divalent cation is applied to the dyed keratinic materials.

A first object of the present disclosure is a process for improving the color retention on keratinous material which has been colored by application of at least one pigment, wherein an aftertreatment agent is applied to the colored keratinous material and optionally rinsed off again after a contact time, wherein the aftertreatment agent is
(N-1) Water,
(N-2) has a pH value of about 7.0 to about 12.5, and
(N-3) comprises at least one salt of a divalent cation.

Work leading to the present disclosure has shown that keratin fibers, particularly hair, could be intensely colored by the application of pigments. Particularly intensive dyeing results with already quite good wash fastnesses were obtained when dyeing was carried out with a combination of pigment and amino silicone. The wash fastness of the dyeings obtained in this way could be further improved if an alkaline after-treatment agent were applied to the hair after dyeing. Due to its content of at least one salt of a divalent cation, the post-treatment agent was able to fix the pigments or the pigments embedded in a film of amino silicone to the hair in a durable manner.

### Keratinic Material

Keratinous material includes hair, skin, nails (such as fingernails and/or toenails). Wool, furs and feathers also fall under the definition of keratinous material. Preferably, keratinous material is understood to be human hair, human skin and human nails, especially fingernails and toenails. Keratinous material is understood to be human hair.

### Coloring Agent

The term “coloring agent” is used in the context of this present disclosure for a coloring of the keratin material, in particular the hair, caused using pigments. In this coloring process, the pigments are deposited as coloring compounds in a particularly homogeneous, uniform and smooth film on the surface of the keratin material.

### Process for Improving Color Retention

For the purposes of the present disclosure, an improvement in color retention is understood to mean an improvement in wash fastness, i.e., with the use of the process as contemplated herein, fewer pigments are removed from the hair in subsequent hair washes. Color retention can be quantified, for example, by colorimetric measurements (measurement of L, a, b values) and calculation of the color distance. The smaller the color gap between washed and unwashed hair, the better the wash fastness or color retention.

### Amino Functionalized Silicone Polymer in the Colorant

The aftertreatment agent used in the process as contemplated herein showed a particularly strong effect when a combination of pigments with amino silicones was used in the preceding dyeing step of the keratin material or keratin fibers.

In the context of a very particularly preferred embodiment, a process as contemplated herein is therefore wherein the aftertreatment agent is applied to keratin material which has been colored by application of at least one amino-functionalized silicone polymer and at least one pigment.

The amino-functionalized silicone polymer may alternatively be referred to as amino silicone or amodimethicone.

Silicone polymers are macromolecules with a molecular weight of at least about 500 g/mol, preferably at least about 1000 g/mol, more preferably at least about 2500 g/mol, particularly preferably at least about 5000 g/mol, which comprise repeating organic units.

The maximum molecular weight of the silicone polymer depends on the degree of polymerization (number of polymerized monomers) and the batch size and is also partly determined by the polymerization method. For the purposes of the present disclosure, it is preferred if the maximum molecular weight of the silicone polymer is not more than about 10⁷ g/mol, preferably not more than about 10⁶ g/mol, and particularly preferably not more than about 10⁵ g/mol.

The silicone polymers comprise many Si—O repeating units, and the Si atoms may carry organic radicals such as alkyl groups or substituted alkyl groups. Alternatively, a silicone polymer is therefore also referred to as polydimethylsiloxane.

Corresponding to the high molecular weight of silicone polymers, these are based on more than about 10 Si—O repeat units, preferably more than about 50 Si—O repeat units, and more preferably more than about 100 Si—O repeat units, most preferably more than about 500 Si—O repeat units.

An amino-functionalized silicone polymer is understood to be a functionalized silicone that carries at least one structural unit with an amino group. Preferably, the amino-functionalized silicone polymer carries multiple structural units, each having at least one amino group. An amino group is understood to mean a primary amino group, a secondary amino group and a tertiary amino group. All these amino groups can be protonated in the acidic environment and are then present in their cationic form.

In principle, good dyeing performance could be achieved with amino-functionalized silicone polymers if they carry at least one primary, at least one secondary and/or at least one tertiary amino group. However, intense colorations with the best wash fastness were obtained when an amino-functionalized silicone polymer comprising at least one secondary amino group was used in the agent.

In a very particularly preferred embodiment, a process as contemplated herein is wherein the aftertreatment agent is applied to keratin material which has been colored by application of at least one amino-functionalized silicone polymer having at least one secondary amino group.

The secondary amino group(s) may be located at various positions on the amino-functionalized silicone polymer. Particularly good color results were obtained when an amino-functionalized silicone polymer was used which has at least one, preferably several, structural units of the formula (Si-Amino).

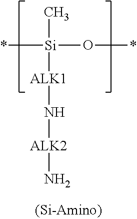

In the structural units of the formula (Si amino), the abbreviations ALK1 and ALK2 independently stand for a linear or branched, bivalent C₁-C₂₀ alkylene group.

In another very particularly preferred embodiment, a process as contemplated herein is wherein the aftertreatment agent is applied to keratin material that has been colored by application of at least one amino-functionalized silicone polymer comprising at least one structural unit of the formula (Si-Amino),

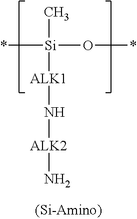

where

ALK1 and ALK2 independently represent a linear or branched C₁-C₂₀ divalent alkylene group.

The positions marked with an asterisk (*) indicate the bond to further structural units of the silicone polymer. For example, the silicon atom adjacent to the star may be bonded to another oxygen atom, and the oxygen atom adjacent to the star may be bonded to another silicon atom or even to a C₁-C₆ alkyl group.

A bivalent C₁-C₂₀ alkylene group can alternatively be referred to as a divalent or divalent C₁-C₂₀ alkylene group, by which is meant that each ALK1 or AK2 grouping can form two bonds.

In the case of ALK1, one bond occurs from the silicon atom to the ALK1 grouping, and the second bond is between ALK1 and the secondary amino group.

In the case of ALK2, one bond is from the secondary amino group to the ALK2 grouping, and the second bond is between ALK2 and the primary amino group.

Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of about 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In another particularly preferred embodiment, the structural units of the formula (Si amino) represent repeat units in the amino-functionalized silicone polymer, such that the silicone polymer comprises multiple structural units of the formula (Si amino).

Particularly well-suited amino-functionalized silicone polymers with at least one secondary amino group are listed below.

Dyeings with the best wash fastnesses could be obtained if, during the preceding dyeing, at least one agent comprising at least one amino-functionalized silicone polymer comprising structural units of formula (Si-I) and formula (Si-II) was applied to the keratinous material

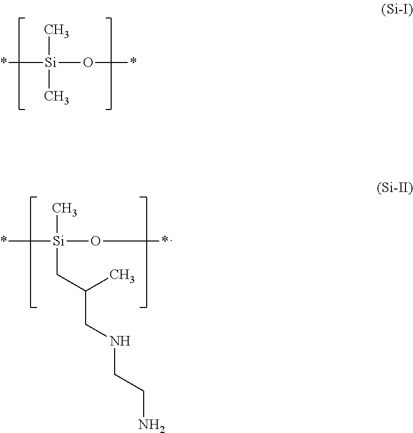

In another explicitly very particularly preferred embodiment, a process as contemplated herein is wherein the aftertreatment agent is applied to keratin material which has been colored by application of at least one amino-functionalized silicone polymer, which comprises structural units of the formula (Si-I) and of the formula (Si-II)

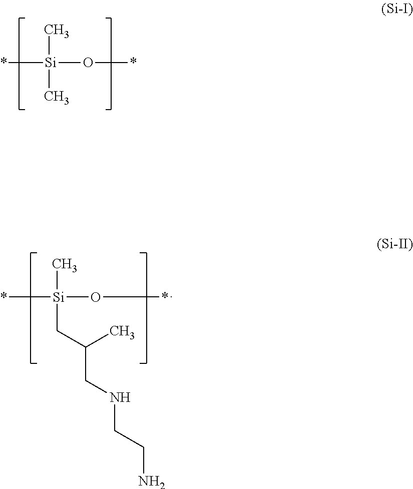

A corresponding amino functionalized silicone polymer with the structural units (Si-I) and (Si-II) is, for example, the commercial product DC 2-8566 or Dowsil 2-8566 Amino Fluid, which is commercially distributed by the Dow Chemical Company and bears the designation “Siloxanes and Silicones, 3-[(2-aminoethyl)amino]-2-methylpropyl Me, Di-Me-Siloxane” and the CAS number 106842-44-8. Another particularly preferred commercial product is Dowsil AP-8658 Amino Fluid, which is also sold commercially by the Dow Chemical Company.

In another preferred embodiment, the application of the post-treatment agent may also be carried out on keratin material previously colored by the application of a colorant comprising at least one amino-functional silicone polymer of the formula of formula (Si-III),

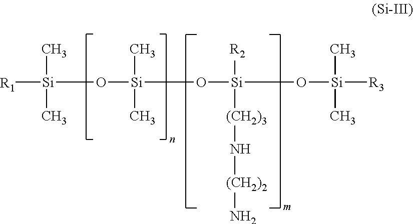

where
m and n mean numbers chosen so that the sum (n+m) is in the range of 1 to about 1000,
n is a number in the range 0 to about 999 and m is a number in the range of 1 to 1 about 000,
R1, R2 and R3, which are the same or different, denote a hydroxy group or a C1-4 alkoxy group,
wherein at least one of R1 to R3 represents a hydroxy group;

Further processes preferred as contemplated herein are exemplified by the prior application of a colorant to the keratinous material, the colorant comprising at least amino-functional silicone polymer of the formula of formula (Si-IV),

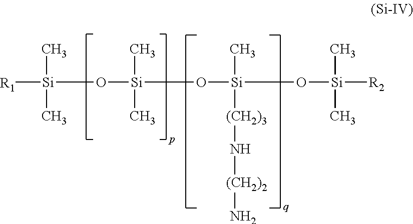

located in the
p and q mean numbers chosen so that the sum (p+q) is in the range of 1 to about 1000,
p is a number in the range of 0 to about 999 and q is a number in the range of 1 to about 1000,
R1 and R2, which are different, denote a hydroxy group or a C1-4 alkoxy group, at least one of R1 to R2 denoting a hydroxy group.

The silicones of the formulas (Si-III) and (Si-IV) differ in the grouping at the Si atom, which carries the nitrogen-comprising group: In formula (Si-III), R2 represents a hydroxy group or a C1-4 alkoxy group, while the radical in formula (Si-IV) is a methyl group. The individual Si groupings, which are marked with the indices m and n or p and q, do not have to be present as blocks; rather, the individual units can also be present in a statistically distributed manner, i.e. in the formulas (Si-III) and (Si-IV), not every R1-Si(CH₃)₂ group is necessarily bonded to an —[O—Si(CH₃)₂] grouping.

Processes as contemplated herein in which a colorant comprising at least one amino-functional silicone polymer of the formula (Si-V) is applied to the keratin fibers have also proved to be particularly effective in producing intense color results.

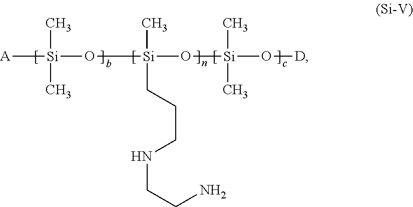

located in the
A represents a group —OH, —O—Si(CH₃)₃, —O—Si(CH₃)₂OH, —O—Si(CH₃)₂OCH₃,
D represents a group —H, —Si(CH₃)₃, —Si(CH₃)₂OH, —Si(CH₃)₂OCH₃,
b, n and c stand for integers between 0 and 1000,
with the specifications
n>0 and b+c>0
   at least one of the conditions A=—OH or D=—H is fulfilled.

In the above formula (Si-V), the individual siloxane units are statistically distributed with the indices b, c and n, i.e., they do not necessarily have to be block copolymers.

The previously applied colorant may further comprise one or more different amino-functionalized silicone polymers represented by the formula (Si-VI)

M(RₐQ_{b}SiO_{(4-a-b)/2)x}(R_{c}SiO_{(4-c)/2)y}M (Si-VI)

in which formula above R is a hydrocarbon or a hydrocarbon radical having from 1 to about 6 carbon atoms, Q is a polar radical of the general formula —R¹HZ wherein R¹ is a divalent linking group bonded to hydrogen and the radical Z composed of carbon and hydrogen atoms, carbon, hydrogen and oxygen atoms, or carbon, hydrogen and nitrogen atoms, and Z is an organic amino functional radical comprising at least one amino functional group; “a” takes values ranging from about 0 to about 2, “b” takes values ranging from about 1 to about 3, “a”+“b” is less than or equal to 3, and “c” is a number ranging from about 1 to about 3, and x is a number ranging from 1 to about 2,000, preferably from about 3 to about 50 and most preferably from about 3 to about 25, and y is a number in the range of from about 20 to about 10,000, preferably from about 125 to about 10,000 and most preferably from about 150 to about 1,000, and M is a suitable silicone end group as known in the prior art, preferably trimethylsiloxy. Non-limiting examples of radicals represented by R include alkyl radicals, such as methyl, ethyl, propyl, isopropyl, isopropyl, butyl, isobutyl, amyl, isoamyl, hexyl, isohexyl and the like; alkenyl radicals, such as vinyl, halovinyl, alkylvinyl, allyl, haloallyl, alkylallyl; cycloalkyl radicals, such as cyclobutyl, cyclopentyl, cyclohexyl and the like; phenyl radicals, benzyl radicals, halohydrocarbon radicals, such as 3- chloropropyl, 4-bromobutyl, 3,3,3-trifluoropropyl, chlorocyclohexyl, bromophenyl, chlorophenyl and the like, and sulfur-comprising radicals, such as mercaptoethyl, mercaptopropyl, mercaptohexyl, mercaptophenyl and the like; preferably R is an alkyl radical comprising from 1 to about 6 carbon atoms, and most preferably R is methyl. Examples of R¹ include methylene, ethylene, propylene, hexamethylene, decamethylene, —CH₂CH(CH₃)CH₂ phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic amino functional radical comprising at least one amino functional group. One formula for Z is NH(CH₂)_{z}NH₂, where z is 1 or more. Another formula for Z is —NH(CH₂)_{z}(CH₂)_{zz}NH, wherein both z and zz are independently 1 or more, this structure comprising diamino ring structures, such as piperazinyl. Z is most preferably an —NHCH₂CH₂NH₂ radical. Another formula for Z is —N(CH₂)_{z}(CH₂)_{zz}NX₂ or —NX₂, wherein each X of X₂ is independently chosen from hydrogen and alkyl groups having 1 to about 12 carbon atoms, and zz is 0.

Q is most preferably a polar, amine-functional radical of the formula —CH₂CH₂CH₂NHCH₂CH₂NH₂. In the formulas, “a” takes values ranging from about 0 to about 2, “b” takes values ranging from about 2 to about 3, “a”+“b” is less than or equal to 3, and “c” is a number ranging from about 1 to about 3. The molar ratio of RₐQ_{b}SiO_{(4-a-b)/2} units to R_{c}SiO_{(4-c)/2} units is in the range of about 1:2 to about 1:65, preferably from about 1:5 to about 1:65 and most preferably by about 1:15 to about 1:20. If one or more silicones of the above formula are used, then the various variable substituents in the above formula may be different for the various silicone components present in the silicone mixture.

In a particularly preferred embodiment, a process as contemplated herein is exemplified by the prior application of a colorant to the keratinous material, said colorant comprising an amino-functional silicone polymer of formula (Si-VII)

R′ₐG₃₋ₐ₋Si(OSiG₂)ₙ-(OSiG_{b}R′_{2-b})ₘ—O—SiG₃₋ₐ₋R′ₐ (Si-VII),

wherein:
G is —H, a phenyl group, —OH, —O—CH₃, —CH₃, —O—CH₂CH₃, —CH₂CH₃, CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, CH(CH₃)CH₂CH₃, —O—C(CH₃)₃, —C(CH3)₃;
a stands for a number between 0 and about 3, especially 0;
b stands for a number between 0 and about 1, especially 1,
m and n are numbers whose sum (m+n) is between 1 and about 2000, preferably between about 50 and about 150, where n preferably assumes values from 0 to about 1999 and from about 49 to about 149 and m preferably assumes values from about 1 to about 2000, from about 1 to about 10,

R′ is a monovalent radical selected from
-Q-N(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H2A⁻,
   where each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
   R″ represents identical or different radicals chosen from —H, -phenyl, -benzyl, —CH₂—CH(CH₃)Ph, the C₁₋₂₀ alkyl radicals, preferably —CH₃, —CH₂CH₃, —CH₂CH₂CH₃, —CH(CH₃)₂, —CH₂CH₂CH₂H3, —CH₂CH(CH₃)₂, —CH(CH₃)CH₂CH₃, —C(CH₃)₃, and A represents an anion preferably selected from chloride, bromide, iodide or methosulfate.

In the context of a further preferred embodiment, a process as contemplated herein is exemplified by the prior application of a colorant to the keratinous material, the colorant comprising at least one amino-functional silicone polymer of the formula (Si-VIIa),

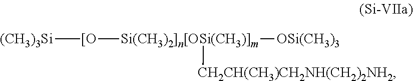

wherein m and n are numbers whose sum (m+n) is between about 1 and about 2000, preferably between about 50 and about 150, n preferably assuming values from 0 to about 1999 and from about 49 to about 149, and m preferably assuming values from about 1 to about 2000, from about 1 to about 10.

According to the INCI declaration, these silicones are called trimethylsilylamodimethicones.

In a further preferred embodiment, a process as contemplated herein is exemplified by the prior application of a colorant to the keratinous material, said colorant comprising at least one amino-functional silicone polymer of formula (Si-VIIb)

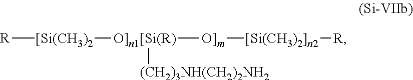

in which R represents —OH, —O—CH₃ or a —CH₃ group and m, n1 and n2 are numbers whose sum (m+n1+n2) is between about 1 and about 2000, preferably between about 50 and about 150, the sum (n1+n2) preferably assuming values from 0 to about 1999 and from about 49 to about 149 and m preferably assuming values from about 1 to about 2000, from about 1 to about 10.

According to the INCI declaration, these amino-functionalized silicone polymers are called amodimethicones.

Regardless of which amino functional silicones are used, colorants as contemplated herein are preferred which contain an amino functional silicone polymer whose amine number is above about 0.25 meq/g, preferably above about 0.3 meq/g and above about 0.4 meq/g. The amine number represents the milliequivalents of amine per gram of the amino-functional silicone. It can be determined by titration and expressed in the unit mg KOH/g.

Furthermore, colorants which included a special 4-morpholinomethyl-substituted silicone polymer are also suitable for use in the process as contemplated herein. This amino-functionalized silicone polymer comprises structural units of the formulae (SI-VIII) and of the formula (Si-IX)

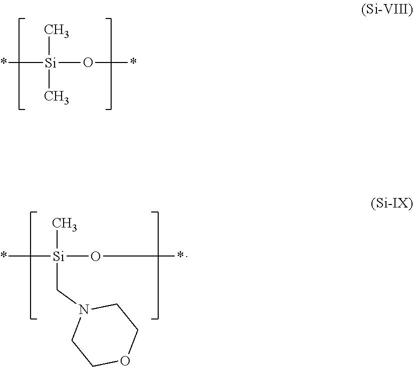

Corresponding 4-morpholinomethyl-substituted silicone polymers are described below.

A corresponding amino-functionalized silicone polymer is available under the name of Amodimethicone/Morpholinomethyl Silsesquioxane Copolymer is known and commercially available from Wacker in the form of the raw material Belsil ADM 8301 E.

As a 4-morpholinomethyl-substituted silicone, for example, a silicone can be used which has structural units of the formulae (Si-VIII), (Si-IX) and (Si-X)

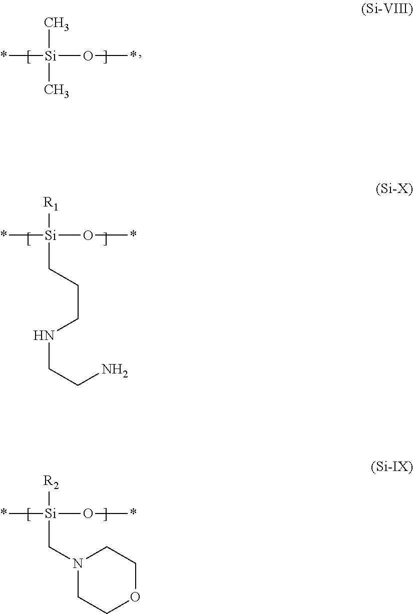

in which
R1 is —CH₃, —OH, —OCH₃, —O—CH₂CH₃, —O—CH₂CH₂CH₃, or —O—CH(CH₃)₂;
R2 is —CH₃, —OH, or —OCH₃.
Particularly preferred colorants contain at least one 4-morpholinomethyl-substituted silicone of the formula (Si-XI)

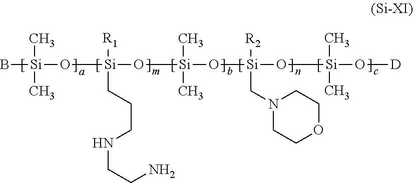

located in the
R1 is —CH₃, —OH, —OCH₃, —O—CH₂CH₃, —O—CH₂CH₂CH₃, or —O—CH(CH₃)₂;
R2 is —CH₃, —OH, or —OCH₃.
B represents a group —OH, —O—Si(CH₃)₃, —O—Si(CH₃)₂OH, —O—Si(CH₃)₂OCH₃,
D represents a group —H, —Si(CH₃)₃, —Si(CH₃)₂OH, —Si(CH₃)₂OCH₃,
a, b and c stand independently for integers between 0 and about 1000, with the condition a+b+c>0
m and n independently of each other stand for integers between 1 and about 1000

with the proviso that
at least one of the conditions B=—OH or D=—H is fulfilled,
the units a, b, c, m and n are distributed statistically or blockwise in the molecule.

Structural formula (Si-XI) is intended to illustrate that the siloxane groups n and m do not necessarily have to be directly bonded to a terminal grouping B or D, respectively. Rather, in preferred formulas (Si-VI) a>0 or b>0 and in particularly preferred formulas (Si-VI) a>0 and c>0, i.e., the terminal grouping B or D is preferably attached to a dimethylsiloxy grouping. Also, in formula (Si-VI), the siloxane units a, b, c, m and n are preferably statistically distributed.

The silicones used as contemplated herein represented by formula (Si-VI) can be trimethylsilyl-terminated (D or B=—Si(CH₃)₃), but they can also be dimethylsilylhydroxy-terminated on two sides or dimethylsilylhydroxy-terminated and dimethylsilylmethoxy-terminated on one side. Silicones particularly preferred in the context of the present disclosure are selected from silicones in which

B=—O—Si(CH₃)₂OH and D=−Si(CH₃)₃
B=—O—Si(CH₃)₂OH and D=−Si(CH₃)₂OH
B=—O—Si(CH₃)₂OH and D=−Si(CH₃)₂OCH₃
B=−O—Si(CH₃)₃ and D=−Si(CH₃)₂OH
B=—O—Si(CH₃)₂OCH₃ and D=−Si(CH₃)₂OH
to everyone. These silicones lead to exorbitant improvements in the hair properties of the hair treated with the agents of the present disclosure, and to a seriously improved protection in oxidative treatment.

The colorants used in the preceding coloring step may contain one or more amino-functionalized silicone polymers, for example, in a total amount of from 0.1 to 8.0 wt. %, preferably from 0.2 to 5.0 wt. %, more preferably from 0.3 to 3.0 wt. %, and most preferably from 0.4 to 2.5 wt. %. Here, the quantities are based on the total quantity of all amino silicones used, which is set in relation to the total weight of the colorant.

In the context of a further particularly preferred embodiment, a process as contemplated herein is wherein the colorant comprises—based on the total weight of the agent—one or more amino-functionalized silicone polymers in a total amount of from about 0.1 to about 8.0 wt. %, preferably from about 0.4 to about 5.0 wt. %, more preferably from about 0.8 to about 3.0 wt. % and very particularly preferably from about 1.0 to about 3.5 wt. %.

### Pigments in the Colorant

In the process as contemplated herein, a post-treatment agent is applied to keratin material that has previously been colored by applying at least one pigment.

Pigments within the meaning of the present disclosure are coloring compounds which have a solubility in water at 25° C. of less than about 0.5 g/L, preferably less than about 0.1 g/L, even more preferably less than about 0.05 g/L. Water solubility can be determined, for example, by the method described below: about 0.5 g of the pigment are weighed in a beaker. A stir-fish is added. Then one liter of distilled water is added. This mixture is heated to about 25° C. for one hour while stirring on a magnetic stirrer. If undissolved components of the pigment are still visible in the mixture after this period, the solubility of the pigment is below about 0.5 g/L. If the pigment-water mixture cannot be assessed visually due to the high intensity of the finely dispersed pigment, the mixture is filtered. If a proportion of undissolved pigments remains on the filter paper, the solubility of the pigment is below about 0.5 g/L.

Suitable color pigments can be of inorganic and/or organic origin. In a preferred embodiment, a process as contemplated herein is wherein the aftertreatment agent is applied to keratin material that has been colored by application of at least one inorganic and/or organic pigment.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

As contemplated herein, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a preferred embodiment, a process as contemplated herein is wherein the aftertreatment agent is applied to keratin material which has been colored by application of at least one inorganic pigment, the inorganic pigment preferably being chosen from colored metal oxides, metal hydroxides, metal oxide hydrates, silicates, metal sulfides, complex metal cyanides, metal sulfates, bronze pigments and/or mica- or mica-based colored pigments coated with at least one metal oxide and/or a metal oxychloride.

In a preferred embodiment, a process as contemplated herein is wherein the aftertreatment agent is applied to keratin material which has been colored by application of at least one pigment selected from mica- or mica-based pigments which have been colored with one or more metal oxides chosen from titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In another embodiment, the previously applied colorant may also contain one or more organic pigments.

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a process as contemplated herein, wherein the aftertreatment agent is applied to keratin material which has been colored by application of at least one organic pigment, wherein the organic pigment is preferably chosen from carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above mentioned conditions. The particles can, for example, be inorganic substrates, which can be aluminum, silica, calcium borosilate, calcium aluminum borosilicate or even aluminum.

For example, alizarin color varnish can be used.

Due to their excellent light and temperature resistance, the use of the above pigments in the agent is particularly preferred. It is also preferred if the pigments used have a certain particle size. As contemplated herein, it is therefore advantageous if the at least one pigment has an average particle size D₅₀ of about 1.0 to about 50 μm, preferably about 5.0 to about 45 μm, preferably about 10 to about 40 μm, about 14 to about 30 μm. The average particle size D₅₀, for example, can be determined using dynamic light scattering (DLS).

In the agent used for the preceding coloration in the process as contemplated herein, one or more pigments may be present, for example, in a total amount of from about 0.01 to about 10.0 wt. %, preferably from about 0.1 to about 5.0 wt. %, more preferably from about 0.2 to about 2.5 wt. % and very particularly preferably from about 0.25 to about 1.5 wt. %. Here, the quantities are based on the total quantity of all pigments used, which is set in relation to the total weight of the colorant.

In another very particularly preferred embodiment, a colorant as contemplated herein is wherein the colorant comprises—based on the total weight of the colorant—one or more pigments in a total amount of from about 0.01 to about 10.0 wt. %, preferably from about 0.1 to about 5.0 wt. %, more preferably from about 0.2 to about 2.5 wt. % and very particularly preferably from about 0.25 to about 1.5 wt. %.

As a further optional component, the colorants could also additionally contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than about 0.5 g/L and are therefore not to be regarded as pigments.

Preferably, the direct dyes within the meaning of the present disclosure have a solubility in water (760 mmHg) at 25° C. of more than about 1.0 g/L.

Direct dyes can be divided into anionic, cationic and non-ionic direct dyes.

In a further embodiment, an agent as contemplated herein may be wherein it additionally comprises at least one colorant compound chosen from anionic, nonionic and cationic direct dyes.

Suitable cationic direct dyes include Basic Blue 7, Basic Blue 26, HC Blue 16, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76.

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—COO⁻, —SO₃⁻present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. In this disclosure, dyes can also be used in the form of their sodium salts and/or their potassium salts.

The acid dyes within the meaning of the present disclosure have a solubility in water (760 mmHg) at 25° C. of more than about 0.5 g/L and are therefore not to be regarded as pigments. Preferably the acid dyes within the meaning of the present disclosure have a solubility in water (760 mmHg) at 25° C. of more than about 1.0 g/L.

The alkaline earth salts (such as calcium salts and magnesium salts) or aluminum salts of acid dyes often have a lower solubility than the corresponding alkali salts. If the solubility of these salts is below about 0.5 g/L (25° C., 760 mmHg), they do not fall under the definition of a direct dye.

An essential characteristic of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In a further embodiment, an agent for dyeing keratinous material may be wherein it comprises at least one anionic direct dye chosen from the nitrophenylenediamines, the nitroaminophenols, the azo dyes, the anthraquinone dyes, the triarylmethane dyes, the xanthene dyes the rhodamine dyes, the oxazine dyes and/or the indophenol dyes, the dyes from the abovementioned group each having at least one carboxylic acid group (—COOH), a sodium carboxylate group (—COONa), a potassium carboxylate group (—COOK), a sulfonic acid group (—SO₃H), a sodium sulfonate group (—SO₃Na) and/or a potassium sulfonate group (—SO₃K).

Suitable acid dyes may include, for example, one or more compounds selected from the following group: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Real red D, FD&C Red Nr. 2, Food Red 9, Naphthol red S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I. 42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. About 0.1 g of the anionic direct dye is placed in a beaker. A stir-fish is added. Then add about 100 ml of water. This mixture is heated to 25° C. on a magnetic stirrer while stirring. It is stirred for about 60 minutes. The aqueous mixture is then visually assessed. If there are still undissolved radicals, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If about 0.1 g of the anionic direct dye dissolves in about 100 ml water at 25° C., the solubility of the dye is about 1.0 g/L.

Acid Yellow 1 is called 8-hydroxy-5,7-dinitro-2-naphthalenesulfonic acid disodium salt and has a solubility in water of at least about 40 g/L (25° C.).

Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above about 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than about 7 g/L (25° C.).
Acid Red 18 is the trinatrium salt of 7-hydroxy-8-[(E)-(4-sulfonato-1-naphthyl)-diazenyl)]-1,3-naphthalene disulfonate and has a remarkably high water solubility of more than about 20 wt. %.
Acid Red 33 is the dinatrium salt of 5-amino-4-hydroxy-3-(phenylazo)-naphthalene-2,7-disulphonate, its solubility in water is about 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).

Acid Blue 9 is the disodium salt of 2-({4[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a solubility in water of more than about 20 wt. % (25° C.).

In a further embodiment, a colorant as contemplated herein is therefore wherein it comprises at least one direct dye chosen from Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct-acting dye or dyes can be used in various amounts in the colorant, depending on the desired color intensity. Satisfactory results could be obtained if the colorant—based on the total weight of the colorant—comprises one or more direct dyes in a total amount of about 0.01 to about 10.0 wt. %, preferably about 0.1 to about 8.0 wt. %, more preferably about 0.2 to about 6.0 wt. % and most preferably about 0.5 to about 4.5 wt. %.

Furthermore, the agent may also contain, as an additional optional component, a coloring compound chosen from photochromic or thermochromic dyes.

Photochromic dyes are dyes that react to irradiation with UV light (sunlight or black light) with a reversible change in hue. In this process, the UV light changes the chemical structure of the dyes and thus their absorption behavior (photochromism).

Thermochromic dyes are dyes that react to temperature changes with a reversible change in hue. In this process, the change in temperature alters the chemical structure of the dyes and thus their absorption behavior (Thermochromism).

The colorant may contain—based on the total weight of the colorant—one or more photochromic and/or thermochromic dyes in a total amount of from about 0.01 to about 10.0 wt. %, preferably from about 0.1 to about 8.0 wt. %, more preferably from about 0.2 to about 6.0 wt. % and most preferably from about 0.5 to about 4.5 wt. %.

### After-Treatment Agent

In the process as contemplated herein, a post-treatment agent is applied to the keratin material, to human hair, which has been dyed as described above. The after-treatment agent is applied to the dyed keratin material and can then be rinsed off again after a contact time, if necessary. This application of the after-treatment agent is associated with a significant improvement in color retention.

The time when the after-treatment product is applied depends on the needs of the user and can be adapted to his habits.

For example, it is possible to apply the aftertreatment agent to the freshly dyed, still wet or moist keratin material, so that a period of only a few minutes to several hours lies between the rinsing out of the dyeing agent and the application of the aftertreatment agent. Furthermore, the user can also decide to dye the hair the day before and apply the after-treatment product only the next day. In this case, the after-treatment product can be applied to the already colored, dry or previously moistened hair.

Likewise, it is possible that there is a longer period between the previous application of the colorant and the application of the post-treatment agent, which can range from a few to several days. This may be the case, for example, if the after-treatment agent is made up in the form of a shampoo to be applied at the time when the user would next wash his hair.

When applying the post-treatment agent, the general requirement is that the post-treatment agent is applied to colored keratin material, which means that the keratin material must still be colored by the application of the pigments.

In one embodiment, the post-treatment agent as contemplated herein can be applied to the colored keratin material and not rinsed out again. In this case, the post-treatment agent usually remains on the keratin material, especially on the hair, until the next wash. When the aftertreatment agent is applied to hair, it can be made up, for example, in the form of a hair tonic, a styling agent or a gel or foam that does not need to be rinsed out.

However, in the context of a further embodiment, it may also be of advantage to rinse out the post-treatment agent as contemplated herein again after an exposure time. Here, the exposure time can be, for example, about 15 seconds to about 15 minutes, preferably about 15 seconds to about 10 minutes, and particularly preferably about 15 seconds to about 5 minutes.

Very preferably, the aftertreatment agent is provided to the user in the form of a shampoo, which is rinsed out of the keratin material or hair again with water after the exposure time.

Very particularly preferred is a process for improving the color retention on keratinous material which has been colored by application of at least one amino-functionalized silicone polymer and at least one pigment, wherein an aftertreatment agent is applied to the colored keratinous material and is rinsed off again after an exposure time of about 15 seconds to about 5 minutes, wherein the aftertreatment agent is
(N-1) Water,
(N-2) has a pH value of about 7.0 to about 12.5, and
(N-3) comprises at least one salt of a divalent cation.

The aftertreatment agent is wherein it comprises water (N-1), has a pH of about 7.0 to about 12.5 (N-2) and comprises at least one salt of a divalent cation (N-3).

### Water Content in the Aftertreatment Agent

The aftertreatment agent comprises water (N-1) or comprises an aqueous carrier. The presence of water in the post-treatment agent allows the salts of the divalent cations (N-3) to be dissolved as completely as possible. To ensure complete dissolution of the salts, the water content in the post-treatment agent is preferably set to a specific range of values.

It has been found preferable if the water content (N-1) in the aftertreatment agent —based on the total weight of the aftertreatment agent—was in the range from about 50 to about 99 wt. %, preferably from about 55 to about 98 wt. %, more preferably from about 60 to about 97 wt. %, and particularly preferably from about 70 to about 96 wt. %.

In a further preferred embodiment, a process as contemplated herein is therefore wherein the aftertreatment agent—based on the total weight of the aftertreatment agent—has a water content (N-1) of from about 50 to about 99 wt. %, preferably from about 55 to about 98 wt. %, more preferably from about 60 to about 97 wt. %, and particularly preferably from about 70 to about 96 wt. %.

### pH Value in the Aftertreatment Agent

The second essential feature of the present disclosure in the aftertreatment agent is its pH (N-2). In this context, it was observed that optimum color retention could only be achieved with an alkaline aftertreatment agent. For this reason, an aftertreatment agent as contemplated herein has a pH of at least about 7. Preferably, the pH value is in a range above about 7.

Particularly beneficial effects were observed when the post-treatment agent has a pH in the range of about 7.5 to about 12.0, preferably about 8.0 to about 11.5, more preferably about 8.0 to about 10.0, and most preferably about 8.0 to about 9.0.

In another particularly preferred embodiment, a process as contemplated herein is therefore wherein the post-treatment agent has a pH (N-2) of from about 7.5 to about 12.0, preferably from about 8.0 to about 11.5, more preferably from about 8.0 to about 10.0, and most preferably from about 8.0 to about 9.0.

In principle, the pH values described above can be adjusted by using the usual alkalizing agents known and approved by the skilled person in the cosmetics field.

Suitable alkalizing agents can thus be, for example, ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Alkanolamines particularly preferred as contemplated herein are selected from 2-aminoethan-1-ol and/or 2-amino-2-methylpropan-1-ol, arginine, lysine, ornithine, histidine, sodium hydroxide, and potassium hydroxide.

However, to produce particularly good color retention, it has proved particularly advantageous to use the salts of divalent cations (N-3), which dissolve in water with an alkaline pH, to adjust the alkaline pH. Explicitly quite particularly preferred, the pH values (N-2) as contemplated herein are therefore adjusted with magnesium hydroxide and/or calcium hydroxide.

Accordingly, a very particularly preferred process for improving the color retention on keratinous material which has been colored by the application of at least one pigment, wherein an aftertreatment agent is applied to the colored keratinous material and is rinsed off again after an exposure time of about 15 seconds to about 5 minutes, wherein the aftertreatment agent is
(N-1) Water comprises,
(N-2) has a pH of from about 7.5 to about 12.0, preferably from about 8.0 to about 11.5, more preferably from about 8.0 to about 10.0, and
(N-3) comprises at least one salt of a divalent cation, where the pH was adjusted by the salt of the divalent cation (N-3).

Explicitly quite particularly preferred is also a process for improving the color retention on keratinous material which has been colored by application of at least one amino-functionalized silicone polymer and at least one pigment, wherein an aftertreatment agent is applied to the colored keratinous material and is rinsed off again after an exposure time of about 15 seconds to about 5 minutes, wherein the aftertreatment agent is
(N-1) Water comprises,
   (N-2) has a pH of from about 7.5 to about 12.0, preferably from about 8.0 to about 11.5, more preferably from about 8.0 to about 10.0, and
   (N-3) comprises calcium hydroxide and/or magnesium hydroxide.

### Salts of Divalent Cations in the Aftertreatment Agent

As an essential component of the present disclosure, the aftertreatment agents used in the process as contemplated herein contain at least one salt of a divalent cation (N-3).

Bivalent cations are defined as cations with a double positive charge. Examples of bivalent cations are Ca²⁺, Mg²⁺, Ba²⁺, Cu²⁺, Fe²⁺ and Zn²⁺.

In a further embodiment, a process as contemplated herein is wherein the post-treatment agent comprises at least one salt (N-3) from the group comprising calcium salts, magnesium salts, barium salts, copper(II) salts, iron(II) salts and zinc(II) salts.

Within the group of the salts, the calcium salts and the magnesium salts are preferred.

In a further embodiment, a process as contemplated herein is wherein the post-treatment agent comprises at least one salt (N-3) from the group of the calcium salts and/or the magnesium salts.

In the salts of divalent cations, the two positive charges are neutralized by the presence of the corresponding equivalents of anionically charged counterions. The anions present as counterions can be inorganic or organic counterions.

Examples of organic anionic counterions are citrates, lactates, malonates, maleate, benzoates and tartrates.

For example, an aftertreatment agent as contemplated herein may contain at least one salt of a divalent cation (N-3) chosen from calcium citrate, calcium lactate, calcium malonate, calcium maleate, calcium benzoate, calcium tartrate, magnesium citrate, calcium lactate, calcium malonate, magnesium maleate, calcium benzoate and magnesium tartrate.

Post-treatment agents comprising at least one inorganic salt of a divalent cation are particularly preferred.

Explicitly quite particularly preferred is thus a process for improving the color retention on keratinous material which has been colored by application of at least one amino-functionalized silicone polymer and at least one pigment, wherein an aftertreatment agent is applied to the colored keratinous material and is rinsed off again after an exposure time of about 15 seconds to about 5 minutes, wherein the aftertreatment agent is
(N-1) Water comprises,
   (N-2) has a pH of from about 7.5 to about 12.0, preferably from about 8.0 to about 11.5, more preferably from about 8.0 to about 10.0, and
   (N-3) comprises at least one inorganic salt of a bivalent cation.

In a further embodiment, a process as contemplated herein is wherein the post-treatment agent comprises at least one salt (N-3) from the group of inorganic calcium salts and/or inorganic magnesium salts.

The inorganic salts of calcium and magnesium are particularly preferred.

The particularly well-suited anionic counterions can be selected, for example, from the group of hydroxides, carbonates, hydrogen carbonates, silicates, phosphates, sulfates and hydrogen phosphates.

Particularly good color retention was observed with the aftertreatment agents comprising at least one salt (N-3) chosen from calcium hydroxide, magnesium hydroxide, calcium carbonate, magnesium carbonate, calcium hydrogen carbonate, magnesium carbonate, calcium silicate, magnesium silicate, calcium phosphate, magnesium phosphate, calcium sulfate, magnesium sulfate, calcium hydrogen sulfate and magnesium hydrogen sulfate, calcium chloride, magnesium chloride, calcium bromide, magnesium bromide, calcium fluoride and magnesium fluoride.

In a further particularly preferred embodiment, a device as contemplated herein is The process wherein the aftertreatment agent comprises at least one salt (N-3) chosen from calcium hydroxide, magnesium hydroxide, calcium carbonate, magnesium carbonate, calcium hydrogen carbonate, magnesium hydrogen carbonate, calcium silicate, magnesium phosphate, magnesium phosphate, calcium sulfate, magnesium sulfate, calcium hydrogen sulfate and magnesium hydrogen sulfate.

In the most preferred embodiment, a device as contemplated herein is a The process wherein the aftertreatment agent (N-3) comprises calcium hydroxide and/or magnesium hydroxide.

Calcium hydroxide has the molecular formula Ca(OH)₂ and carries the CAS number 1305-62-0 and is alternatively called slaked lime or hydrated lime.

Magnesium hydroxide has the molecular formula Mg(OH)₂ and carries the CAS number 1309-42-8.
Calcium carbonate has the molecular formula CaCO₃ and carries the CAS number 471-34-1 or the number 13397-26-7 (calcite).
Magnesium carbonate has the molecular formula MgCO3 and carries the CAS number 546-93-0 or 13717-00-5 (monohydrate), 5145-48-2 (dihydrate), 14457-83-1 (trihydrate) or 61042-72-6 (pentahydrate).
Calcium hydrogen carbonate has the molecular formula Ca(HCO₃)₂ and is also known as calcium bicarbonate. Calcium hydrogen carbonate is used in the form of its aqueous solution.
Magnesium carbonate has the molecular formula Mg(HCO₃)₂ and is also used in the form of its aqueous solution.
Calcium silicate has the molecular formula CaSiO3 and carries the CAS number 1344-95-2 or 111811-33-7 (hydrate).
Magnesium silicate is a group of compounds which are the magnesium salts of silicic acids with the empirical formula MgOxSiO2, where x is the average molar ratio of silicon dioxide to magnesium oxide. Under the group of magnesium silicates fall

| | Alternative | | |
|---|---|---|---|
| Name | name | CAS-No | Molecular formula |
| Magnesium | Talcum powder | 14807-96-6 | Mg₃Si₄O₁₀(OH)₂ |
| silicate | | 63210-56-0 | |
| hydrate | | (artificial) | |
| basic | Silicic acid | 35592-05-3 | Mg₃Si₄O₁₀(OH)₂ |
| magnesium | (H₂SiO₃) | | |
| silicate | Magnesium salt | | |
| | (4:3) | | |
| Magnesium | Silica (H₆Si₂O₇) | 15702-53-1 | Mg₃Si₂O₇ |
| silicate | Magnesium salt | | |
| anhydrate | (1:3) | | |
| Magnesium | Silica magnesium | 12263-17-1 | Mg₃Si₂O₇•2 H₂O |
| silicate | salt hydrate | | |
| dihydrate | (1:3:2) | | |
| Magnesium | Silicic acid | 1343-90-4 | MgO•xSiO₂ |
| silicate | magnesium | | (x = 1, 4-4) |
| hydrate | salt hydrate | | |
| Magnesium | Magnesium | 14987-04-3 | Mg₂Si₃O₈ |
| trisilicate | silicon | | |
| anhydrate | dioxide | | |
| Magnesium | Silicic acid | 39365-87-2 | Mg₂Si₃O₈•x H₂O |
| trisilicate | (H₄Si₃O₈) | | |
| hydrate | magnesium salt | | |
| | (1:2) hydrate | | |
| Magnesium | Silicic acid | 10034-94-3 | Mg₂SiO₄ |
| orthosilicate | (H4SiO4) | 26686-77-1 | |
| | Magnesium salt | | |
| | (1:2) | | |
| Magnesium | Silicic acid | 13776-74-4 | MgSiO₃ |
| metasilicate | (H₂SiO₃) | | |
| | Magnesium salt | | |
| | (1:1) | | |
| Magnesium | Silicic acid | 30079-89-1 | MgₓSiO₃ |
| metasilicate | (H₂SiO₃) | | |
| | Magnesium salt | | |
| Chrysotile | | 12001-29-5 | Mg₃Si₂O₅(OH)₄ |
| Antigorite | | 12135-86-3 | Mg₆[(OH)₈\|Si₄O₁₀] |
| Lizardite | | 12161-84-1 | Mg₃Si₂O₅(OH)₄ |

Calcium phosphate has the molecular formula Ca₃(PO₄)₂ and carries the CAS number 7758-87-4.

In food technology, magnesium phosphates are collectively referred to as magnesium dihydrogen phosphate magnesium hydrogen phosphate and magnesium phosphate. They are approved in the European Union as food additives under the common number E 343.
Calcium sulfate has the molecular formula CaSO₄ and carries the CAS number 7778-18-9 (anhydrous) or 10034-76-1 (hemihydrate), 10101-41-4 (dihydrate) or 13397-24-5 (hydrate).
Magnesium sulfate has the molecular formula MgSO₄ and carries the CAS number 7487-88-9 (magnesium sulfate) or 10034-99-8 (MgSO₄-7H₂O).
Calcium hydrogen sulfate has the empirical formula Ca(HSO₄)₂.
Magnesium hydrogen sulfate has the molecular formula Mg(HSO₄)₂.
Calcium chloride has the molecular formula CaCl2 and carries the CAS numbers 10043-52-4 (anhydrous), 13477-29-7 (monohydrate), 10035-04-8 (dihydrate), 25094-02-4 (tetrahydrate), 7774-34-7 (hexahydrate) and 22691-02-7 (hydrate).Magnesium chloride has the molecular formula MgCl2 and carries the CAS numbers 7786-30-3 (anhydrous) and 7791-18-6 (hexahydrate).
Calcium bromide has the molecular formula CaBr₂ and carries the CAS numbers 7789-41-5 (anhydrous), 71626-99-8 (hydrate), 22208-73-7 (dihydrate) and 13477-28-6 (hexahydrate). Magnesium bromide has the molecular formula MgBr2 and carries the CAS numbers 7789-48-2 and 13446-53-2 (hexahydrate).

As described above, it has proved particularly advantageous regarding the solution of the problem as contemplated herein if alkaline-reacting salts are used as salts of the divalent cations (N-3), which are used to adjust the alkaline pH value as contemplated herein.

Accordingly, the post-treatment agent will contain the salt(s) (N-3) in such an amount as is necessary to adjust the required pH (N-2). In the context of this embodiment, the amount of salts (N-3) to be used depends primarily on the pH value to be adjusted.

Accordingly, a very particularly preferred process for improving the color retention on keratinous material which has been colored by the application of at least one amino-functionalized silicone polymer and at least one pigment, wherein an aftertreatment agent is applied to the colored keratinous material and is rinsed off again after an exposure time of about 15 seconds to about 5 minutes, wherein the aftertreatment agent is
(N-1) Water comprises,
   (N-2) has a pH of from about 7.5 to about 12.0, preferably from about 8.0 to about 11.5, more preferably from about 8.0 to about 10.0, and
   (N-3) comprises at least one salt of a divalent cation in an amount necessary to adjust the pH (N-2).

However, in a further embodiment, it may also be preferred to further increase the ionic strength in the post-treatment agent. In principle, salts of divalent cations (N-3) whose solution in water does not lead to an alkaline pH value can also be used.

Therefore, to further increase the amount of salt (N-3) used while maintaining a certain desired pH, it may also be possible to add one or more acidifying agents to the aftertreatment agent in addition to the alkaline reacting salt (N-3).

The acidifying agents may be the acids known to the skilled person and approved in cosmetics, which may be selected, for example, from the group comprising lactic acid, citric acid, malic acid, hydrochloric acid, sulfuric acid, phosphoric acid, tartaric acid, malonic acid, maleic acid, succinic acid, etidronic acid.

In the context of a further embodiment, it may be preferred if the post-treatment agent—based on the total weight of the post-treatment agent—comprises one or more calcium salts and/or magnesium salts (N-3) in a total amount of from about 0.1 to about 10.0 wt. %, preferably from about 0.2 to about 8.0 wt. %, more preferably from about 0.3 to about 6.0 wt. % and most preferably from about 0.4 to about 2.0 wt. %.

In a further embodiment, a process as contemplated herein is wherein the aftertreatment agent comprises—based on the total weight of the aftertreatment agent—one or more calcium salts and/or magnesium salts (N-3) in a total amount of from about 0.1 to about 10.0 wt. %, preferably from about 0.2 to about 8.0 wt. %, more preferably from about 0.3 to about 6.0 wt. % and very preferably from about 0.4 to about 2.0 wt. %.

### Surfactants in the Aftertreatment Agent

As described above, the aftertreatment agent used in the process as contemplated herein is very preferably packaged as a shampoo. To achieve the cleansing effect, the aftertreatment shampoo therefore very particularly preferably additionally comprises at least one surfactant.

In another very particularly preferred embodiment, a process as contemplated herein is wherein the aftertreatment agent comprises at least one cationic, nonionic and/or anionic surfactant.

The term surfactants (T) refer to surface-active substances that can form adsorption layers on surfaces and interfaces or aggregate in bulk phases to form micelle colloids or lyotropic mesophases. A distinction is made between anionic surfactants comprising a hydrophobic radical and a negatively charged hydrophilic head group, amphoteric surfactants, which carry both a negative and a compensating positive charge, cationic surfactants, which have a positively charged hydrophilic group in addition to a hydrophobic radical, and nonionic surfactants, which have no charges but strong dipole moments and are strongly hydrated in aqueous solution.

Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually comprising a hydrocarbon backbone (e.g., comprising one or two linear or branched alkyl chains) and the positive charge(s) being in the hydrophilic head group. Examples of cationic surfactants are
quaternary ammonium compounds which, as hydrophobic radicals, may carry one or two alkyl chains with a chain length of about 8 to about 28 C atoms,
quaternary phosphonium salts substituted with one or more alkyl chains with a chain length of about 8 to about 28 C atoms or
tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of about 0.1 to about 45 wt. %, preferably about 1 to about 30 wt. % and most preferably about 1 to about 15 wt. %—based on the total weight of the respective agent.

Non-ionic surfactants contain, for example, a polyol group, a polyalkylene glycol ether group or a combination of polyol and polyglycol ether group as the hydrophilic group. Such links include
Addition products of about 2 to 5 about 0 mol ethylene oxide and/or 0 to about 5 mol propylene oxide to linear and branched fatty alcohols with about 6 to about 30 C atoms, the fatty alcohol polyglycol ethers or the fatty alcohol polypropylene glycol ethers or mixed fatty alcohol polyethers,
Addition products of about 2 to about 50 mol ethylene oxide and/or 0 to about 5 mol propylene oxide to linear and branched fatty acids with about 6 to about 30 C atoms, the fatty acid polyglycol ethers or the fatty acid polypropylene glycol ethers or mixed fatty acid polyethers,
Addition products of 2 to 50 mol ethylene oxide and/or 0 to about 5 mol propylene oxide to linear and branched alkylphenols having about 8 to about 15 C atoms in the alkyl group, the alkylphenol polyglycol ethers or the alkylpolypropylene glycol ethers or mixed alkylphenol polyethers,
with a methyl or C₂-C₆-alkyl radical end-group capped addition products of about 2 to about 50 moles of ethylene oxide and/or 0 to about 5 moles of propylene oxide to linear and branched fatty alcohols with about 8 to about 30 C atoms, to fatty acids with about 8 to about 30 C atoms and to alkylphenols with about 8 to about 15 C atoms in the alkyl group, such as the grades available under the sales names Dehydol® LS, Dehydol® LT (Cognis),
C₁₂-C₃₀ fatty acid mono- and diesters of addition products of 1 to about 30 mol ethylene oxide to glycerol,
Addition products of about 5 to about 60 mol ethylene oxide to castor oil and hardened castor oil,
Polyol fatty acid esters, such as the commercial product Hydagen® HSP (Cognis) or Sovermol® grades (Cognis),
alkoxylated triglycerides,
alkoxylated fatty acid alkyl esters of the formula (Tnio-1)

R¹C—(OCH₂CHR²)_{w}OR³ (Tnio-1)

in which R¹CO is a linear or branched, saturated and/or unsaturated acyl radical having about 6 to about 22 carbon atoms, R² is hydrogen or methyl, R³ is linear or branched alkyl radicals having 1 to about 4 carbon atoms and w is numbers from 1 to about 20,
amine oxides,
Hydroxy mixed ethers, as described for example in DE-OS 19738866,
Sorbitan fatty acid esters and addition products of ethylene oxide to sorbitan fatty acid esters such as polysorbates,
Sugar fatty acid esters and addition products of ethylene oxide to sugar fatty acid ester,
Addition products of ethylene oxide to fatty acid alkanolamides and fatty amines,
Sugar tensides of the alkyl and alkenyl oligoglycosides type according to formula (E4-II),

R⁴O—[G]ₚ (Tnio-2)

in which R⁴ is an alkyl or alkenyl radical comprising about 4 to about 22 carbon atoms, G is a sugar residue comprising about 5 or about 6 carbon atoms and p is a number of 1 to about 10. They can be obtained by the relevant methods of preparative organic chemistry. The alkyl and alkenyl oligoglycosides can be derived from aldoses or ketoses with 5 or 6 carbon atoms, preferably glucose. The preferred alkyl and/or alkenyl oligoglycosides are thus alkyl and/or alkenyl oligoglucosides. The index number p in the general formula (Tnio-2) indicates the degree of oligomerization (DP), i.e. the distribution of mono- and oligoglycosides and stands for a number between 1 and about 10. While p must always be an integer in the individual molecule and can assume the values p=1 to about 6, the value p for a certain alkyl oligoglycoside is an analytically determined arithmetical quantity, which usually represents a fractional number. Preferably alkyl and/or alkenyl oligoglycosides with an average degree of oligomerization p of about 1.1 to about 3.0 are used. From an application technology point of view, those alkyl and/or alkenyl oligoglycosides are preferred whose degree of oligomerization is less than about 1.7 and lies between about 1.2 and about 1.4. The alkyl or alkenyl radical R⁴ can be derived from primary alcohols comprising about 4 to about 11, preferably about 8 to about 10 carbon atoms. Typical examples are butanol, caproic alcohol, caprylic alcohol, caprin alcohol and undecrylic alcohol as well as their technical mixtures, such as those obtained in the hydrogenation of technical fatty acid methyl esters or during the hydrogenation of aldehydes from Roelen's oxo synthesis. Preferred are alkyl oligoglucosides with a chain length of C₈-C₁₀ (DP=1 to about 3), which are obtained as a preliminary step in the distillative separation of technical C₈-C₁₈ coconut-fatty alcohol and may be contaminated with less than about 6 wt. % of C₁₂ alcohol, and alkyl oligoglucosides based on technical C_{9/11} oxoalcohols (DP=1 to about 3). The alkyl or alkenyl radical R¹⁵ can also be derived from primary alcohols having about 12 to about 22, preferably about 12 to about 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and their technical mixtures, which can be obtained as described above. Preferred are alkyl oligoglucosides based on hardened C12/14 coconut alcohol with a DP of 1 to about 3.
Sugar surfactants of the fatty acid N-alkyl polyhydroxyalkylamide type, a nonionic surfactant of formula (Tnio-3)

R⁵CO—NR⁶—[Z] (Tnio-3)

in which R⁵CO is an aliphatic acyl radical comprising about 6 to about 22 carbon atoms, R⁶ is hydrogen, an alkyl or hydroxyalkyl radical comprising 1 to about 4 carbon atoms and [Z] is a linear or branched polyhydroxyalkyl radical comprising about 3 to about 12 carbon atoms and about 3 to about 10 hydroxyl groups. The fatty acid N-alkyl polyhydroxyalkylamides are known substances that can usually be obtained by reductive amination of a reducing sugar with ammonia, an alkylamine or an alkanolamine and subsequent acylation with a fatty acid, a fatty acid alkyl ester or a fatty acid chloride. The fatty acid N-alkyl polyhydroxyalkylamides are preferably derived from reducing sugars with 5 or 6 carbon atoms, especially from glucose. The preferred fatty acid N-alkyl polyhydroxyalkylamides are therefore fatty acid N-alkylglucamides as represented by the formula (Tnio-4):

R⁷CO—(NR⁸)—CH₂—[CH(OH)]₄—CH₂OH (Tnio-4)

Preferably, glucamides of the formula (Tnio-4) are used as fatty acid-N-alkyl polyhydroxyalkylamides, in which R⁸ represents hydrogen or an alkyl group and R⁷CO represents the acyl radical of caproic acid, caprylic acid, capric acid, Lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or their technical mixtures. Particularly preferred are fatty acid N-alkyl glucamides of the formula (Tnio-4), which are obtained by reductive amination of glucose with methylamine and subsequent acylation with lauric acid or C12/14 coconut fatty acid or a corresponding derivative. Furthermore, polyhydroxyalkylamides can also be derived from maltose and palatinose.

Other typical examples of nonionic surfactants are fatty acid amide polyglycol ethers, fatty amine polyglycol ethers, mixed ethers or mixed formals, protein hydrolysates (especially wheat-based vegetable products) and polysorbates.

The alkylene oxide addition products to saturated linear fatty alcohols and fatty acids, each with 2 to 30 moles of ethylene oxide per mole of fatty alcohol or fatty acid, and the sugar surfactants have proved to be preferred nonionic surfactants. Preparations with excellent properties are also obtained if they contain fatty acid esters of ethoxylated glycerol as non-ionic surfactants.

Furthermore, the post-treatment agents as contemplated herein may also contain at least one anionic surfactant. Anionic surfactants are surface-active agents with exclusively anionic charges (neutralized by a corresponding counter cation). Examples of anionic surfactants are fatty acids, alkyl sulphates, alkyl ether sulphates and ether carboxylic acids with about 12 to about 20 C atoms in the alkyl group and up to about 16 glycol ether groups in the molecule.

The surfactants described above are preferably used in the post-treatment agent in the appropriate quantity ranges. Thus, based on the total weight of the aftertreatment agent, the aftertreatment agent may contain one or more nonionic surfactants in a total amount of from about 0.1 to about 20 wt. %, preferably from about 0.2 to about 15 wt. %, more preferably from about 0.3 to about 10 wt. %.

Grease component in the aftertreatment agent

As an optional ingredient, the aftertreatment agent may also contain one or more fat components.

The fatty components are hydrophobic substances that can form emulsions in the presence of water, forming micelle systems.

For the purposes of the present disclosure, “fatty components” means organic compounds with a solubility in water at room temperature (22° C.) and atmospheric pressure (760 mmHg) of less than about 1 wt. %, preferably less than about 0.1 wt. %. The definition of fat constituents explicitly covers only uncharged (i.e., non-ionic) compounds. Fat components have at least one saturated or unsaturated alkyl group with at least about 12 C atoms. The molecular weight of the fat constituents is a maximum of about 5000 g/mol, preferably a maximum of about 2500 g/mol and particularly preferably a maximum of about 1000 g/mol. The fat components are neither polyoxyalkylated nor polyglycerylated compounds.

Very preferably, the fat constituents included in the aftertreatment agent are chosen from C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

In the context of a further embodiment, a process as contemplated herein is wherein the aftertreatment agent comprises at least one fat constituent from the group comprising the C₁₂-C₃₀ fatty alcohols, the C₁₂-C₃₀ fatty acid triglycerides, the C₁₂-C₃₀ fatty acid monoglycerides, the C₁₂-C₃₀ fatty acid diglycerides and/or the hydrocarbons.

Preferred fat constituents in this context are the constituents from the group of C₁₂-C₃₀ fatty alcohols. For the purposes of the present disclosure, only non-ionic substances are explicitly regarded as fat constituents. Charged compounds such as fatty acids and their salts are not considered to be fat components. The work leading to the present disclosure has shown that post-treatment agents comprising one or more C₁₂-C₃₀ fatty alcohols are particularly good at removing excess amino silicones or pigments.

The C₁₂-C₃₀ fatty alcohols can be saturated, mono- or polyunsaturated, linear or branched fatty alcohols with about 12 to about 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, Cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol) and/or brassidyl alcohol ((13E)-docosen-1-ol).

The preferred representatives for branched fatty alcohols are 2-octyl-dodecanol, 2-hexyl-dodecanol and/or 2-butyl-dodecanol.

In one embodiment, good results were obtained when the post-treatment agent comprises one or more C₁₂-C₃₀ fatty alcohols chosen from dodecan-1-ol (dodecyl alcohol, lauryl alcohol), Tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol), Behenyl alcohol (docosan-1-ol), (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), Gadoleyl alcohol ((9Z)-Eicos-9-en-1-ol), Arachidone alcohol ((5Z,8Z,11Z,14Z)-Eicosa-5,8,11,14-tetraen-1-ol), Erucyl alcohol ((13Z)-Docos-13-en-1-ol), Brassidyl alcohol ((13E)-docosen-1-ol) 2-octyl-dodecanol, 2-hexyl-dodecanol and/or 2-butyl-dodecanol.

In another embodiment, a process as contemplated herein is wherein the post-treatment agent comprises one or more C₁₂-C₃₀ fatty alcohols chosen from

Dodecan-1-ol (dodecyl alcohol, lauryl alcohol),
Tetradecan-1-ol (tetradecyl alcohol, myristyl alcohol),
Hexadecan-1-ol (hexadecyl alcohol, cetyl alcohol, palmityl alcohol),
Octadecan-1-ol (octadecyl alcohol, stearyl alcohol),
Arachyl alcohol (eicosan-1-ol),
Heneicosyl alcohol (heneicosan-1-ol),
Behenyl alcohol (docosan-1-ol),
(9Z)-Octadec-9-en-1-ol (oleyl alcohol),
(9E)-Octadec-9-en-1-ol (elaidyl alcohol),
(9Z,12Z)-Octadeca-9,12-dien-1-ol (linoleyl alcohol),
(9Z,12Z,15Z)-Octadeca-9,12,15-trien-1-ol (linolenoyl alcohol),
Gadoleyl alcohol ((9Z)-Eicos-9-en-1-ol),
Arachidonic alcohol ((5Z,8Z,11Z,14Z)-Eicosa-5,8,11,14-tetraen-1-ol),
Erucyl alcohol ((13Z)-docos-13-en-1-ol),
Brassidyl alcohol ((13E)-docosen-1-ol),

### 2-Octyl-dodecanol,

2-hexyl dodecanol and/or
2-Butyl-dodecanol comprises.

Furthermore, it has proven particularly preferable to use one or more C₁₂-C₃₀ fatty alcohols in specific ranges of amounts in the aftertreatment agent.

It is particularly preferred if the post-treatment agent comprises one or more C₁₂-C₃₀ fatty alcohols in a total amount of from about 0.1 to about 12.0 wt. %, preferably from about 0.5 to about 10.0 wt. %, more preferably from about 1.0 to about 8.0 wt. %, based on the total weight of the post-treatment agent.

Further, as a suitable fat ingredient, the agent may also contain at least one C₁₂-C₃₀ fatty acid triglyceride that is C₁₂-C₃₀ fatty acid monoglyceride and/or C₁₂-C₃₀ fatty acid diglyceride. For the purposes of the present disclosure, a C₁₂-C₃₀ fatty acid triglyceride is understood to be the triester of the trivalent alcohol glycerol with three equivalents of fatty acid. Both structurally identical and different fatty acids within a triglyceride molecule can be involved in the formation of esters.

As contemplated herein, fatty acids are to be understood as saturated or unsaturated, unbranched or branched, unsubstituted or substituted C₁₂-C₃₀ carboxylic acids. Unsaturated fatty acids can be mono- or polyunsaturated. For an unsaturated fatty acid, its C—C double bond(s) may have the Cis or Trans configuration.

Fatty acid triglycerides are exemplified by suitability in which at least one of the ester groups is formed starting from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

The fatty acid triglycerides can also be of natural origin. The fatty acid triglycerides or mixtures thereof occurring in soybean oil, peanut oil, olive oil, sunflower oil, macadamia nut oil, moringa oil, apricot kernel oil, marula oil and/or optionally hydrogenated castor oil are suitable for use in the product as contemplated herein.

A C₁₂-C₃₀ fatty acid monoglyceride is understood to be the monoester of the trivalent alcohol glycerol with one equivalent of fatty acid. Either the middle hydroxy group of glycerol or the terminal hydroxy group of glycerol may be esterified with the fatty acid.

C₁₂-C₃₀ fatty acid monoglycerides are particularly suitable in which a hydroxyl group of glycerol is esterified with a fatty acid, the fatty acids being selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

A C₁₂-C₃₀ fatty acid diglyceride is the diester of the trivalent alcohol glycerol with two equivalents of fatty acid. Either the middle and one terminal hydroxy group of glycerol may be esterified with two equivalents of fatty acid, or both terminal hydroxy groups of glycerol are esterified with one fatty acid each. The glycerol can be esterified with two structurally identical fatty acids or with two different fatty acids.

Fatty acid diglycerides are exemplified by suitability in which at least one of the ester groups is formed starting from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

It is further as contemplated herein if the aftertreatment agent included at least one C₁₂-C₃₀ fatty acid monoglyceride selected from the monoesters of glycerol with one equivalent of fatty acid from the group comprising dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), Petroselic acid [(Z)-6-octadecenoic acid], Palmitoleic acid [(9Z)-Hexadec-9-enoic acid], Oleic acid [(9Z)-Octadec-9-enoic acid], Elaidic acid [(9E)-Octadec-9-enoic acid], Erucic acid [(13Z)-Docos-13-enoic acid], Linoleic acid [(9Z, 12Z)-Octadeca-9, 12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid].

In the context of a further embodiment, a process as contemplated herein is wherein the post-treatment agent comprises at least one C₁₂-C₃₀ fatty acid monoglyceride selected from the monoesters of glycerol with one equivalent of fatty acid chosen from dodecanoic acid, tetradecanoic acid, hexadecanoic acid, tetracosanoic acid, octadecanoic acid, eicosanoic acid and/or docosanoic acid.

Further, as quite suitable fatty ingredients, the aftertreatment agent may also contain at least one hydrocarbon.

Hydrocarbons are compounds comprising exclusively of the atoms carbon and hydrogen with 8 to 80 C atoms. In this context, aliphatic hydrocarbons such as mineral oils, liquid paraffin oils (e.g., Paraffinum Liquidum or Paraffinum Perliquidum), isoparaffin oils, semi-solid paraffin oils, paraffin waxes, hard paraffin (Paraffinum Solidum), Vaseline and polydecenes are particularly preferred.

Liquid paraffin oils (Paraffinum Liquidum and Paraffinum Perliquidum) have proven to be particularly suitable in this context. Paraffinum Liquidum, also known as white oil, is the preferred hydrocarbon. Paraffinum Liquidum is a mixture of purified, saturated, aliphatic hydrocarbons, comprising hydrocarbon chains with a C-chain distribution of about 25 to about 35 C-atoms.

Further work has also shown that beneficial effects could be achieved when the fatty components described above, especially the C₁₂-C₃₀ fatty alcohols described as particularly suitable, are used in combination with at least one ester oil in the aftertreatment agent as contemplated herein.

Ester oils are esters of C₁₂-C₃₀ fatty acids with aliphatic C₁-C₂₄ alcohols that have a liquid aggregate state at room temperature (25° C.). In other words, ester oils as contemplated herein are exemplified by having a melting point below 25° C. at normal pressure (1013 mbar).

It has been found to be preferable when an after-treatment agent was applied to the previously colored hair, which included at least one ester oil chosen from the monoesters of C₁₂-C₂₄ fatty acids with aliphatic monohydric C₁-C₂₄ alcohols.

In a further embodiment, the process as contemplated herein is wherein the aftertreatment agent comprises at least one fatty constituent chosen from esters of a C₁₂-C₃₀ fatty acid and an aliphatic monohydric C₁-C₂₄ alcohol.

Within the group of C₁₂-C₃₀ fatty acids, the C₁₂-C₂₄ fatty acids are particularly well suited. Examples of C₁₂-C₂₄ fatty acids suitable for forming the ester oils are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid, and their technical mixtures. Examples of the fatty alcohol portions in the ester oils include isopropyl alcohol, caprylic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical mixtures thereof.

These C₁₂-C₂₄ fatty acids are esterified by reaction with a C₁-C₂₄ aliphatic alcohol, which is particularly preferably a mono-alcohol, so that the esterification produces a mono-ester.

The C₁-C₂₄ aliphatic alcohols can be linear or branched, saturated, or mono- or polyunsaturated.

For example, an alcohol chosen from methanol, ethanol, n-propanol, iso-propanol, n-butanol, n-pentanol, 2-ethyl-hexanol, n-hexanol, n-octanol, n-decanol and n-dodecanol can be used as the C₁-C₂₄ aliphatic saturated alcohol.

Examples of monovalent, unsaturated, C₁-C₂₄ alcohols include oleyl alcohol (octadec-9-en-1-ol), Palmitoleyl alcohol (cis-9-Hexadecen-1-ol), elaidyl alcohol (trans-9-Octadecen-1-ol) and cis-11-Octadecen-1-ol.

To form the esters as contemplated herein, the C₁₂-C₂₄ fatty acids and the C₁-C₁₂ alcohols are selected so that the ester formed by esterification from the two reactants is an ester oil, i.e., it has a melting point below about 25° C. at about 1013 mbar.

Some ester oils as contemplated herein can be used in the form of commercially available raw materials, which are mixtures of the esters obtained from fatty acids of different chain length and/or alcohols of different chain length. These raw materials may have a melting range. For these raw materials, a melting point below about 25° C. means that the melting process begins at a temperature below about 25° C.

If, for example, an ester oil in the form of a certain raw material can be used in the agent, this raw material having a melting range of about 16 to about 27° C., then this raw material comprises at least one ester oil having a melting point below about 25° C. Thus, this ester oil is as contemplated herein.

Particularly preferred as contemplated herein are 2-ethylhexyl palmitate (Cegesoft® 24), isopropyl myristate (Rilanit® IPM), isononanoic acid C16-18 alkyl ester (Cetiol® SN), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), cetearyl isononanoate (Cetiol® SN), oleic acid decyl ester (Cetiol® V).

Most preferably, the ester oil is chosen from isopropyl myristate, 2-ethylhexyl palmitate, isononanoic acid C16-18 alkyl ester, stearic acid 2-ethylhexyl ester, cetyloleate, coconut fatty alcohol caprinate, coconut fatty alcohol caprylate, n-butyl stearate, oleyl erucate, isopropyl palmitate, oleyl oleate, lauric acid hexyl ester, cetearyl isononanoate and oleic acid decyl ester.

In the context of a further embodiment, a process as contemplated herein is wherein the aftertreatment agent comprises at least one ester oil chosen from isopropyl myristate, 2-ethylhexyl palmitate, isononanoic acid C16-18 alkyl ester, stearic acid 2-ethylhexyl ester, cetyloleate, coconut fatty alcohol caprinate, coconut fatty alcohol caprylate, n-butyl stearate, oleyl erucate, isopropyl palmitate, oleyl oleate, lauric acid hexyl ester, cetearyl isononanoate and oleic acid decyl ester.

Isopropyl myristate is alternatively known as myristic acid isopropyl ester and has CAS number 110-27-0. Isopropyl myristate is a colorless and odorless liquid. The melting point is bsi about 0-1° C.

2-Ethylhexyl palmitate is alternatively known as hexadecanoic acid 2-ethylhexyl ester and has CAS number 29806-73-3⋅2-Ethylhexyl palmitate is a branched, saturated ester oil of palmitic acid and ethyl hexyl alcohols. 2-Ethylhexyl palmitate is present at room temperature in the form of a clear, colorless liquid that has a slightly fatty odor.

Isononanoic acid C16-18 alkyl ester is alternatively called cetearyl isononanoate, this ester has the CAS numbers 84878-33-1 and 84878-34-2. Isononanoic acid C16-18 alkyl ester is a clear, slightly yellowish liquid. At 20° C., isononanoic acid C16-18 alkyl ester has a viscosity of about 19-22 mPas.

Stearic acid 2-ethylhexyl ester is alternatively known as ethylhexyl stearate and has the CAS number 91031-48-0. Stearic acid 2-ethylhexyl ester is in the form of a clear, slightly yellowish, thin liquid oil. At 20° C., stearic acid 2-ethylhexyl ester has a viscosity of about 14-16 mPas and is more of an oil at room temperature.

Cetyloleate has the CAS number 22393-86-8.

Coconut fatty alcohol caprylate/caprate carries the CAS number 95912-86-0. Is a mixture of C8-C10 fatty acids with C12-C18 fatty alcohols, which is in the form of a yellow liquid and which has a melting point of about 10° C.

n-Butyl stearate is also known alternatively as stearic acid butyl ester and has the CAS numbers 85408-76-0 (C16-18) and 123-95-5 (C18). n-Butyl stearate is a yellowish liquid and begins to melt at about 16° C.

Oleyl erucate carries the CAS number 17673-56-2. Oleylerucate is a yellow liquid. At about 20° C., oleyl acrylate has a viscosity of about 40-50 mpas and is therefore an oil at room temperature.

Isopropyl palmitate is also known alternatively as propan-2-yl hex decanoate and has the CAS number 142-91-6. The melting point of isopropyl palmitate is about 13.5° C.

Oleyl oleate is alternatively known as cis-9,10-octadecenyl cis-9,10-octadecanoate or oleic acid oleyl ester and has CAS number 3687-45-4. Oleyl oleate is a clear, slightly yellowish oil that has a viscosity of about 25-30 mPas at about 20° C. and is an oil at room temperature.

Lauric acid hexyl ester is alternatively known as hexyl laurate and has the CAS number 34316-64-8. Lauric acid hexyl ester is a clear, yellowish, odorless oil at room temperature. At about 20° C., lauric acid hexylester has a viscosity of about 5-7 mpas and is more of an oil at room temperature.

Cetearyl isononanoate is alternatively known as isononanoic acid C16-18 alkyl ester and has CAS numbers 84878-33-1 and 84878-34-2. Cetearyl Isononanoate is a yellowish liquid with a melting point of about 16-22° C.

Oleic acid decyl ester is alternatively known as decyl oleate and has the CAS number 3687-46-5. Oleic acid decyl ester is a slightly yellowish liquid that has a viscosity of about 15-20 mPas at about 20° C. Accordingly, oleic acid decyl ester is an oil at room temperature.

Satisfactory results were obtained when the aftertreatment agent included—based on the total weight of the agent—one or more ester oils in a total amount of about 0.1 to about 10.0 wt. %, preferably about 0.2 to about 7.0 wt. %, more preferably about 0.3 to about 5.0 wt. % and most preferably about 0.4 to about 1.5 wt. %.

In a further embodiment, a process as contemplated herein is wherein the aftertreatment agent—based on the total weight of the aftertreatment agent—comprises one or more fat constituents in a total amount of about 0.1 to about 10.0 wt. %, preferably about 0.5 to about 8.0 wt. %, preferably about 1.5 to about 6.5 wt. % and most preferably about 1.8 to about 4.5 wt. %.

### Other Optional Ingredients in the Aftertreatment Agent

In addition to the essential ingredients of the present disclosure already described, the aftertreatment agent may also contain other optional ingredients, such as solvents, anionic, nonionic, zwitterionic and/or cationic polymers; structurants such as glucose, maleic acid and lactic acid, hair-conditioning compounds such as phospholipids, for example lecitin and cephalins; perfume oils, dimethyl isosorbide and cyclodextrins; fiber structure-improving agents, in particular mono-, di- and oligosaccharides such as glucose, galactose, fructose, fructose and lactose; dyes for coloring the agent; antidandruff agents such as piroctone olamine, zinc omadine and climbazole; amino acids and oligopeptides; protein hydrolysates on animal and/or vegetable basis, as well as in the form of their fatty acid condensation products or, optionally, anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; Polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; Fats and waxes such as fatty alcohols, beeswax, montan wax and kerosene; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescing agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. Regarding other optional components and the quantities of these components used, explicit reference is made to the relevant manuals known to the specialist. The additional active ingredients and auxiliary substances are preferably used in the preparations as contemplated herein in quantities of about 0.0001 to about 25 wt. % each, about 0.0005 to about 15 wt. %, based on the total weight of the respective agent.

### Process for Dyeing and Post-Treatment of Keratinous Material, Especially Human Hair

As previously described, the time at which the after-treatment agent is applied to the colored hair can be freely chosen depending on the preferences of the user. It can be particularly convenient for the user to carry out the coloring of the hair and the application of the after-treatment agent in directly successive steps within one application process.

A second object of the present disclosure is therefore a process for coloring keratinous fibers, in particular human hair, comprising the following steps:

(1) Applying a colorant to the keratinous fibers, the colorant comprising at least one amino-functionalized silicone polymer and at least one pigment as already disclosed in detail in the description of the first subject matter of the present disclosure,
(2) Exposure of the colorant applied in step (1) to the keratinous fibers,
(3) Rinse out the dye with water,
(4) Applying a post-treatment agent to the dyed keratinous fibers, the post-treatment agent having already been disclosed in detail in the description of the first subject matter of the present disclosure,
(5) Allowing the post-treatment agent applied in step (4) to act on the keratinous fibers, and
(6) If necessary, rinse out the after-treatment agent with water.

In step (1) of the process as contemplated herein, a colorant comprising at least one amino-functionalized silicone polymer and at least one pigment, their preferred and especially preferred representatives described above, is applied to the hair.

In the following step, the previously applied dye to the hair left to act.

In this context, different exposure times of, for example, about 30 seconds to about 60 minutes are conceivable.

However, a major advantage of the dyeing system as contemplated herein is that an intensive color result can be achieved even in short periods after short exposure times. For this reason, it is advantageous if the application mixture remains on the keratin material only for comparatively short periods of time after its application, from about 30 seconds to about 15 minutes, preferably from about 30 seconds to about 10 minutes, and particularly preferably from about 1 to about 5 minutes.

In a further preferred embodiment, a method as contemplated herein is. exemplified by:

(2) Exposure of the colorant applied in step (1) to the keratinous fibers for a period ranging from about 30 seconds to about 15 minutes, preferably from about 30 seconds to about 10 minutes, and more preferably from about 1 to about 5 minutes

Finally, following the action of the dye on the keratin material, it is rinsed with water in step (3). Here, in a preferred embodiment, the colorant is washed out with water only, i.e., without the aid of an after-treatment agent or shampoo not as contemplated herein.

Subsequently, the application of the post-treatment agent, especially in its preferred and particularly preferred embodiments described above, is carried out in step (4).

In this context, it has been found to be particularly preferable to apply the after-treatment agent to the dyed hair when the user would normally perform the first hair wash after dyeing. This is usually done within a period of about 1 to about 3 days after staining, i.e., between steps (3) and (4) of the process there is a maximum period of about 72 hours in this case.

Preferably, a process for coloring keratinous fibers, in particular human hair, comprises the following steps:

(1) Applying a colorant to the keratinous fibers, the colorant comprising at least one amino-functionalized silicone polymer and at least one pigment as already disclosed in detail in the description of the first subject matter of the present disclosure,
(2) Exposure of the colorant applied in step (1) to the keratinous fibers,
(3) Rinse out the dye with water,
(4) Applying an aftertreatment agent to the dyed keratinous fibers within a period not exceeding 72 hours after step (3), the aftertreatment agent having already been disclosed in detail in the description of the first subject matter of the present disclosure,
(5) Allowing the post-treatment agent applied in step (4) to act on the keratinous fibers, and
(6) If necessary, rinse out the after-treatment agent with water.

The action of the post-treatment agent on the keratinous fibers in step (5) may be for a period ranging from about 15 seconds to about 10 minutes, for example, and preferably for a period ranging from about 30 seconds to about 5 minutes.

After that, the post-treatment agent is finally rinsed with water in step (6). Here, in a preferred embodiment, the aftertreatment agent is washed out with water only, i.e., without the aid of an aftertreatment agent not as contemplated herein or another shampoo.

Preferably, a process for coloring keratinous fibers, in particular human hair, comprises the following steps in the order indicated:

(1) Applying a colorant to the keratinous fibers, the colorant comprising at least one amino-functionalized silicone polymer and at least one pigment as already disclosed in detail in the description of the first subject matter of the present disclosure,
(2) Exposure of the colorant applied in step (1) to the keratinous fibers,
(3) Rinse out the dye with water,
(4) Applying a post-treatment agent to the dyed keratinous fibers, the post-treatment agent having already been disclosed in detail in the description of the first subject matter of the present disclosure,
(5) Allowing the post-treatment agent applied in step (4) to act on the keratinous fibers, and
(6) Rinse out the after-treatment agent with water.

In another preferred embodiment, a process as contemplated herein is wherein the post-treatment agent is left to act for a period of about 15 seconds to about 5 minutes and then rinsed with water.

Furthermore, it is quite particularly preferred if the user uses the after-treatment agent as contemplated herein as the shampoo with which he usually washes his hair, i.e., the user then uses the shampoo repeatedly as part of his usual cleaning routine. In this case, steps (4) to (6) of the process are repeated.

### Multi-Component Packaging Unit

To increase user convenience, the user is preferably provided with all the necessary agents in the form of a multi-component packaging unit (kit-of-parts).

A second object of the present disclosure is therefore a multi-component packaging unit (kit-of-parts) for dyeing and post-treatment of keratinous fibers, in particular human hair, comprising separately prepared
a first container comprising a colorant, the colorant comprising at least one amino-functionalized silicone polymer and at least one pigment as already disclosed in detail in the description of the first subject matter of the present disclosure, and
a second container having an after-treatment agent, the after-treatment agent having already been disclosed in detail in the description of the first subject matter of the present disclosure.

Concerning the further preferred embodiments of the multicomponent packaging unit as contemplated herein, mutatis mutandis what has been said about the processes as contemplated herein applies.

### EXAMPLES

### 1. Formulations

The following formulations were prepared (all figures in wt. % unless otherwise stated).

| Colorant (C) | C |
|---|---|
| Cetyl alcohol | 3.0 g |
| Stearyl alcohol | 3.0 g |
| Ceteareth-30, (Cetearyl alcohol, ethoxylated 30 EO) | 1.5 g |
| Potassium hydroxide, 50% aqueous solution | 0.5 g |
| Unipure Red LC 3079, organic pigment CI 15850 | 1.0 g |
| 1.2-propanediol | 10.0 g |
| Dow Corning 2-8566 (siloxanes and silicones, 3-[(2- | 1.0 g |
| aminoethyl)amino]-2-methylpropyl Me, di-Me-siloxane. | |
| Water | ad 100 |

| | PTA-V | PTA-E |
|---|---|---|
| Post-treatment agent (PTA) | Comparison | Inventive |
| Sodium Laureth Sulfate (2 EO) | 5.5 | 5.5 |
| Disodium cocoamphodiacetate | 2.0 | 2.0 |
| Cocoamidopropylbetaine | 1.4 | 1.4 |
| PEG-7 glycerin coconut oil | 0.7 | 0.7 |
| PEG-40 Hydrogenated Castor Oil | 0.3 | 0.3 |
| Benzophenone-4 | 0.1 | 0.1 |
| Apricot kernel oil | 0.1 | 0.1 |
| Polyquatemium-10 | 0.6 | 0.6 |
| D-Panthenol | 0.2 | 0.2 |
| Sodium benzoate | 0.5 | 0.5 |
| Lactic acid | 2.79 | 2.79 |
| Calcium hydroxide Ca(OH)₂ | ad pH 4.5 | ad pH 8.5 |
| Water | ad 100 | ad 100 |

### 2. Application of the Dyeing Agent and the After-Treatment Agent

After production, the colorant (C) was applied to hair strands. The dye was left to act for three minutes. Subsequently, the hair strand was washed thoroughly (1 minute) with water. The strands were dried and then left to rest for 24 hours.

A reference strand was colorimetrically measured using a Datacolor Spectraflash 450 colorimeter.

For the application of the aftertreatment agent, the corresponding strand was moistened with water, then the aftertreatment agent was applied to the strand (0.25 g of aftertreatment agent per 1 g of hair) and massaged in with the fingers for 30 seconds. Then the post-treatment agent was rinsed out for 1 minute under lukewarm running water and the hair strand was dried. The previously described process corresponds to a hair wash. For each subsequent hair wash the procedure was repeated. The hairs after-treated in this way were measured colorimetrically.

### 3. Measurement of the Color Retention

The dE value used to assess color retention is derived from the L*a*b* colorimetric values measured on the respective strand as follows:

dE=[(Lᵢ−L₀)²+(aᵢ−a₀)²+(bᵢ−b₀)]^{1/2}

L₀, a₀ and b₀=measured values of the reference strand (without post-treatment)
Lᵢ, aᵢ and bᵢ=Measured values of the post-treated strand (after 6 hair washes)

The smaller the dE value, the smaller the color gap compared to dyed, non-retouched hair and the better the color retention.

| | L | a | b | dE |
|---|---|---|---|---|
| C without post-treatment | 38.67 | 50.47 | 14.08 | — |
| (0 hair washes) | | | | |
| C + PTA-V (comparison) | 64.25 | 17.04 | 7.00 | 42.7 |
| (After 6 hair washes) | | | | |
| C + PTA-E (Inventive) | 48.93 | 34.06 | 3.62 | 22.0 |
| (After 6 hair washes) | | | | |

dE = color distance compared to the dyed, untreated hair

Post-treatment with the PTA-E after-treatment agent as contemplated herein showed improved color retention.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A process for improving the color retention on keratinous material which has been colored by the application of at least one pigment, wherein an after treatment agent is applied to the colored keratinous material and optionally rinsed off after a reaction time, wherein the after treatment agent comprises
(N-1) Water
(N-2) has a pH value of about 7.0 to about 12.5, and
(N-3) further comprises at least one salt of a divalent cation.

2. The process according to claim 1, wherein the keratinous material is colored by application of at least one amino-functionalized silicone polymer and at least one pigment.

3. The process according to claim 2, wherein the keratinous material is colored by application of at least one amino-functionalized silicone polymer having at least one secondary amino group.

4. The process according to claim 2, wherein the keratinous material is colored by application of at least one amino-functionalized silicone polymer comprising at least one structural unit of the formula (Si-Amino), 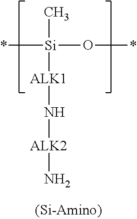
wherein
each of ALK1 and ALK2 independently is a linear or branched C₁-C₂₀ divalent alkylene group.

5. The process according to claim 2, wherein the keratinous material is colored by application of at least one amino-functionalized silicone polymer comprising structural units of formula (Si-I) and formula (Si-II)

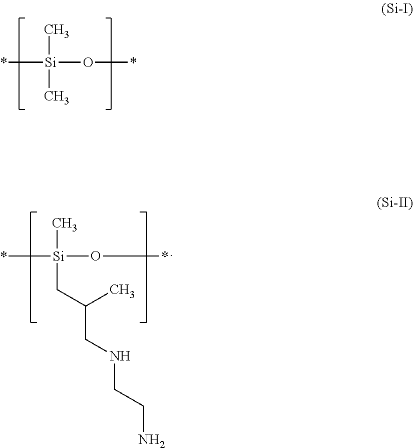

6. The process according to claim 1, wherein the keratinous material is colored by application of at least one inorganic pigment, the inorganic pigment chosen from colored metal oxides, metal hydroxides, metal oxide hydrates, silicates, metal sulfides, complex metal cyanides, metal sulfates, bronze pigments, and/or colored mica- or mica-based pigments coated with at least one metal oxide and/or a metal oxychloride.

7. The process according to claim 1, wherein the keratinous material is colored by application of at least one organic pigment, the organic pigment chosen from carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

8. The process according to claim 1, wherein the after treatment agent—based on the total weight of the after treatment agent—has a water content (N-1) of about 50 to about 99 wt. %.

9. The process according to claim 1, wherein the post-treatment agent has a pH (N-2) of from about 7.5 to about 12.0.

10. The process according to claim 1, wherein the after treatment agent comprises at least one salt (N-3) chosen from calcium salts and/or magnesium salts.

11. The process according to claim 1, wherein the after treatment agent comprises at least one salt (N-3) chosen from calcium hydroxide, magnesium hydroxide, calcium carbonate, magnesium carbonate, calcium hydrogen carbonate, magnesium hydrogen carbonate, calcium silicate, magnesium silicate, calcium phosphate, magnesium phosphate, calcium sulfate, magnesium sulfate, calcium hydrogen sulfate and magnesium hydrogen sulfate, calcium chloride, magnesium chloride, calcium bromide and magnesium bromide.

12. The process according to claim 1, wherein the after treatment agent comprises—based on the total weight of the after treatment agent—one or more calcium salts and/or magnesium salts (N-3) in a total amount of about 0.1 to about 10.0 wt. %.

13. The process according to claim 1, wherein the after treatment agent further comprises at least one cationic, nonionic and/or anionic surfactant.

14. A process for coloring keratinous fibers comprising:
(1) Applying a colorant to the keratinous fibers, the colorant comprising at least one amino-functionalized silicone polymer and at least one pigment,
(2) Exposing the colorant applied in step (1) to the keratinous fibers,
(3) Rinsing the colorant and keratinous fibers with water,
(4) Applying an after treatment agent to the colored keratinous fibers, wherein the after treatment agent has a pH of about 7.0 to about 12.5 and comprises water and at least one salt of a divalent cation,
(5) Allowing the after treatment agent applied in step (4) to act on the keratinous fibers, and
(6) Optionally rinsing the after-treatment agent and the keratinous fibers with water.

15. The process according to claim 1, wherein the after treatment agent is allowed to act for a period of about 15 seconds to about 5 minutes and is then rinsed with water.

16. A multi-component packaging unit (kit-of-parts) for dyeing and post-treatment of keratinous fibers, comprising a separately packaged:
first container comprising a colorant, the colorant comprising at least one amino-functionalized silicone polymer and at least one pigment, and
second container comprising an after treatment agent that has a pH of about 7.0 to about 12.5 and comprises water and at least one salt of a divalent cation.
